(19) United States
(12) Patent Application Publication
(10) Pub. No.: US 2013/0171204 A1
(43) Pub. Date: Jul. 4, 2013

(54) SOFT FOOD COMPOSITION WITH PROBIOTICS AND PREBIOTICS FOR MASKING MEDICATIONS

(71) Applicant: Vets Plus, Inc., Knapp, WI (US)

(72) Inventors: Daniel J. DuBourdieu, Limerick, ME (US); Anita K. Sinha, Menomonie, WI (US); Rajiv Lall, Menomonie, WI (US); Tapan A. Gor, Menomonie, WI (US); Devraj C. Raval, Menomonie, WI (US)

(73) Assignee: VETS PLUS, INC., Knapp, WI (US)

(21) Appl. No.: 13/729,133

(22) Filed: Dec. 28, 2012

Related U.S. Application Data
(60) Provisional application No. 61/581,706, filed on Dec. 30, 2011.

(51) Int. Cl. A61K 9/00; A23K 1/16

(52) U.S. Cl. CPC A61K 9/0056; A23K 1/16
USPC 424/400; 426/61; 426/71; 426/64; 426/63; 424/94.63; 424/94.6; 424/94.61

(57) ABSTRACT

A soft extruded food composition containing probiotics, enzymes, and vitamins for wrapping medications, such as pills or tablets, for animal consumption. The flavor of the wrapped solid medication is masked by the food composition and thus becomes more palatable to the animal while providing additional health benefits in the form of probiotics, prebiotics, enzymes, and vitamins.

### CROSS-REFERENCE TO RELATED APPLICATIONS

This application claims priority under 35 USC §119(e) to U.S. Provisional Patent Application 61/581,706 filed Dec. 30, 2011, the entirety of which is incorporated herein by reference.

### FIELD OF THE INVENTION

The present invention relates to a food product and feed supplements for animals and, more particularly, to a soft food product which can be folded around solid medication to conceal the medication. The invention also relates to food and feed supplements that improve the health of an animal by providing probiotics, prebiotics, vitamins, minerals, and enzymes.

### BACKGROUND

It is difficult to administer pills or other oral medications to animals such as dogs, cats, and horses. In order to administer the medication, the mouth of the animal typically has to be forced open while the medication is inserted. This is very difficult for most animal owners to carry out without injury to themselves or the animal while making sure the medication is actually swallowed. Animal owners have tried to conceal and mask the taste of solid medications by wrapping them in cheese or lunch meat. However, these types of wrappings are not sufficiently sticky to hold onto the medication, and the medication often escapes its wrappings. In addition, these types of wrappings serve as mere carriers and do not have any or have insufficient supplements in the form of probiotics, prebiotics, vitamins, minerals, and enzymes to confer additional health benefits to the animal.

Several attempts at solutions to this problem have been described. U.S. Pat. No. 4,857,333 describes use of a food product matrix with a preformed pocket to conceal medication. However, the pocket tends to get misshaped in the packaging during storage, thus leading to an unusable product. In addition, the food product matrix does not contain any active ingredients that can further benefit the health of the animal.

U.S. Pat. No. 7,067,150 to Farber et al. describes an oral delivery system comprising a matrix which includes at least one starch, one or more carbohydrates, one or more hydrocolloids, one or more sugars, sugar alcohols or sugar syrups, and water. The delivery system is a semi-solid at room temperature and has a final moisture content between about 15% and 30% by weight. It is intended for the administration to both humans and non-humans.

U.S. Pat. No. 5,853,757 to Durand et al. is directed to a soft edible sack which can be used as a carrier for animal medication. The sack is formed from a mixture of dehydrated meat by-product admixed with soy flour and water and a glycerin lubricant. The meat by-product is used to mask the scent of the medicine to make it easier for the animal to swallow the medicine. However, there is no reference to the addition of probiotics, prebiotics, enzymes or vitamins.

U.S. Pat. Nos. 6,905,703 and 7,337,708 to Rothamel et al. are directed to an edible product comprising an edible outer cover encapsulating a medicament. An example is a hairball treatment for cats, where the outer component is a dough-like material. The soft dough material includes flour (soy flour and wheat flour) which can be mixed with other ingredients, such as colors, flavors, vitamins, minerals or other palatants. The dough-like material is cooked and pasteurized and then treated to encapsulate an emulsion for hairball treatment.

U.S Pat. Nos. 6,866,862 and 7,052,712 to Huber et al. are directed to an extruded animal feed which can be semi-moist and can be a “pillow-type” feed having a soft flowable matrix center surrounded by a shell of feed material. An active drug or medication is typically placed in the center. The exterior portion can include flour, such as wheat flour, rice flour or corn flour, corn gluten meal, poultry meal, brewer's yeast, sodium bicarbonate, ethoxyquin, cheese powder and poultry meals. However, probiotics, prebiotics, enzymes, and vitamins are not listed.

U.S. Pat. No. 7,914,816 to Kalbe et al. is directed to a starch-based extruded shaped article which can act as a carrier for pharmaceuticals. The extruded covering typically carries an aroma, such as poultry liver. The ingredients include typical flour products, starches, sugars such as sucrose, glucose and fructose, humectants and plasticizers, emulsifiers and flavoring agents. However, there is no mention of probiotics, prebiotics, vitamins, or enzymes.

Thus, the prior art does not disclose outer composition materials for masking medications that include probiotics, prebiotics, enzymes, and vitamins.

It is therefore an object of the invention to provide a simple method of administrating oral solid medications to animals that allows the medication to be not rejected by the animal.

It is a further object of the invention to add beneficial health ingredients to the composition without the use of added heat as heat may degrade certain ingredients such probiotics and prebiotics.

Another object of the invention is to add these beneficial ingredients to the composition without the solid medication present so the animal may be already used to the delivery system and therefore not reject the medication when they are present in the invention.

### SUMMARY OF THE INVENTION

While there are some products on the market that conceal solid medications to a greater or lesser extent, improvements can be made on the concept. A food composition for animals that contains active ingredients that improve the health of the animal rather than just being used as a treat or a pill carrier is desired. If the composition is regularly given to the animal without the solid medication, the animal will readily accept the composition when a solid medication is inserted.

The present invention is directed to an extruded soft food composition for masking the flavor of solid medications and concealing the solid medications when provided orally to animals by wrapping the solid medication in the soft dough-like food composition.

The food composition includes one, all, or various combinations or subcombinations of the following ingredients: a base powder, an emulsifier, a softening agent, a flavoring, a starch, an antioxidant, a salt with a mono- or divalent cation, a preservative, oil, water, a probiotic, a prebiotic, a digestive enzyme, and a vitamin.

One version of the present invention is a consumable composition for providing health benefits to an animal. The composition comprises a viscoelastic mass that includes a carrier base and an effector component. The carrier base includes one or more components selected from the group consisting of a powder, an emulsifier, a starch, an oil, a softening agent, and water in a combination and in amounts effective to confer viscoelasticity to the viscoelastic mass. The effector component includes one or more components selected from the group consisting of a probiotic, a prebiotic, and an enzyme selected from the group consisting of a carbohydrase, a lipase, and a protease.

In certain versions, the probiotic is a microorganism in a genus selected from the group consisting of Aspergillus, Trichoderma, Bacillus, Bacteriodies, Bifidobacterium, Lactobacillus, Leuconostoc, Streptococcus, Pediococcus, Propionibacterium, Saccharomyces, Enterococcus, Escherichia, and combinations thereof. The probiotic is preferably included in an amount of from about 0.01 to about 1×10¹¹ CFU/g of the viscoelastic mass.

In certain versions, the prebiotic is selected from the group consisting of inulin, lactulose, lactitol, a fructooligosaccharide, a galactooligosaccharide, a xylooligosaccharide, an isomaltooligosaccharide, a mannaoligosaccharide, a lactosucrose, a cereal fiber, a soy oligosaccharide, raffinose, beet pulp, psyllium, cellulose, gum arabic, and combinations thereof. The prebiotic is preferably included in an amount of from about 0.5% to about 6% w/w of the viscoelastic mass.

In certain versions, the composition comprises a carbohydrase. The carbohydrase is preferably selected from the group consisting of a xylanase, a glucanase, and an amylase.

In certain versions, the composition comprises an enzyme. The enzyme is preferably from a fermentation broth of a microorganism in a genus selected from the group consisting of Aspergillus, Trichoderma, Humicola, Candida, Rhizomucor, and Thermomyces. The enzyme is preferably included in an amount of from about 1 unit/lb to about 500 units/lb of the viscoelastic mass.

In certain versions, the composition comprises an enzyme. The enzyme is preferably selected from the group consisting of a B vitamin, vitamin C, vitamin A, vitamin E, vitamin D, and vitamin K. In certain versions, the composition comprises mixed tocopherols. The vitamin is preferably included in an amount of from about 0.01% to about 5% w/w of the viscoelastic mass.

In certain versions, the composition comprises a powder, wherein the powder comprises a plant powder and an animal powder.

In certain versions, the composition comprises an emulsifier, wherein the emulsifier comprises lecithin in an amount of from about 4% to about 16% w/w of the viscoelastic mass.

In certain versions, the composition comprises a softening agent, wherein the softening agent comprises glycerol in an amount of from about 9% to about 14% w/w of the viscoelastic mass.

In certain versions, the composition comprises a preservative in an amount of from about 0.01% to about 3% w/w of the composition, wherein the preservative is selected from the group consisting of potassium sorbate, methylparaben, and propylparaben.

In certain versions, the viscoelastic mass further comprises a flavoring.

In certain versions, the viscoelastic mass is in the form of a pliable sheet.

In certain versions, the composition further comprises a solid or gel disposed within the viscoelastic mass that forms a distinct phase within the viscoelastic mass. The solid or gel is preferably a medication.

In certain versions, the composition further comprises a solid or gel. The viscoelastic mass is in the form of a pliable sheet, and the viscoelastic mass is rolled or folded about the solid or gel.

The advantage to the present invention is that it provides a soft food product for enveloping a solid medication to conceal the medication. In addition, it includes beneficial ingredients, such as probiotics, prebiotics, enzymes, and vitamins.

The objects and advantages of the invention will appear more fully from the following detailed description of the preferred embodiment of the invention made in conjunction with the accompanying drawings.

### DETAILED DESCRIPTION OF THE INVENTION

### Viscoelastic Mass

The composition of the present invention comprises a viscoelastic mass. The viscoelastic mass is an edible, dough-like composition that is capable of being folded, wrapped, or rolled around a medicine or otherwise manipulated to surround the medicine to conceal or mask the flavor of the medicine. The viscoelastic mass comprises a carrier base and one or more effector components.

The carrier base comprises a mixture of components formulated to confer the viscoelasticity to the viscoelastic mass. The carrier base may comprise any or all of the components selected from a base powder, an emulsifier, a starch, an oil, a softening agent, a flavoring, a salt comprising a mono- or divalent cation, a preservative, an antioxidant, and water, among other components known in the art. Suitable exemplary combinations and amounts of such components for conferring viscoelasticity to the carrier base and the viscoelastic mass are described herein.

The effector components comprise components conferring a health benefit to an animal. Examples of effector components include probiotics, prebiotics, enzymes such as digestive enzymes, and vitamins.

### Base Powder

The viscoelastic mass preferably comprises a base powder. The base powder generally provides structural integrity to the mass. The base powder may comprise a plant powder, an animal powder, or both a plant and an animal powder. Plant powders are powders derived from plants, such as flours or other powders. The flours may be whole flours or flours which have had fractions, such as the germ fraction or the husk fraction, removed. Non-limiting examples of suitable plant powders include soy flour, wheat flour, whole wheat flour, whole wheat fine flour, wheat feed flour, wheat gluten, pre-gel wheat flour, soy protein concentrate, oat flour or powder, barley powder or flour, brown rice flour or powder, dried whey powder, carrot powder, cherry powder, pineapple powder, and alfalfa herb powder. Animal powders are powders derived from animals and can include dehydrated meat byproducts, such as liver powder. In a preferred version of the invention, the base powder comprises an animal powder and a plant flour, which can be mixed with a fluid lubricant. The powder is preferably included in an amount of from about 10% to about 70% w/w of the viscoelastic mass.

### Emulsifiers

The viscoelastic mass may include an emulsifier. Suitable emulsifiers include nonionic surfactants, such as polyoxyethylated castor oil, polyoxyethylated sorbitan monooleate, sorbitan monostearate, ethyl alcohol, glycerol monostearate, polyoxyethylene stearate, and alkylphenol polyglycol ethers; ampholytic surfactants, such as disodium N-lauryl-B-iminodipropionate and lecithin; and anionic surfactants, such as sodium lauryl sulphate, fatty alcohol ether sulphates, and mono/dialkyl polyglycol ether orthophosphoric ester monoethanolamine salt. A preferred emulsifier is lecithin, such as soy lecithin. The emulsifier may be included in an amount of from about 0.1% to about 20% w/w of the viscoelastic mass, such as from about 4% to about 16% w/w or from about 6% to about 10% w/w.

### Softening Agents

In order to provide an edible soft chew, the viscoelastic mass preferably includes a softening agent. Examples of suitable softening agents include glycerol and propylene glycol, wetting agents such as cetyl alcohol and glycerol monostearate, and other humectants.

Glycerin is a preferred softening agent and can maintain the softness of the viscoelastic mass over the shelf life of the product. The softening agent may be included in an amount of from about 0.1% to about 50% w/w of the viscoelastic mass, such as from about 5% to about 25% w/w or from about 9% to about 14% w/w.

### Flavorings

A flavoring is preferably included in the viscoelastic mass to enhance the palatability of the mass and to mask the flavor of any medicine included therewith. The flavoring is preferably food grade quality. Sweeteners constitute one type of suitable flavoring. Examples of suitable sweeteners include such sugars as xylose, ribose, sucrose, mannose, galactose, fructose, dextrose, and maltose. Other suitable sweeteners include molasses, honey, maple syrup, and fruit flavoring. The sweeteners may be in powdered, granulated, or liquid form. Natural or synthetic sweeteners are suitable. Preferred sweeteners include powdered sugar and dry molasses. Other suitable flavorings include carob, peanuts, garlic, and herbs such as, parsley, celery, peppermint, and spearmint. Natural and synthetic flavoring oils can also be included as a flavoring. Examples of flavoring oils include anise oil, spearmint oil, peppermint oil, cinnamon oil, wintergreen oil, citrus oils, such as lemon, orange, grape, lime, and grapefruit oils. Other suitable flavorings include fruit essences such as apple, strawberry, cherry, and pineapple essences, among others. The flavoring may be included in an amount of from about 0.1% to about 40% w/w of the viscoelastic mass, such as from about 2% to about 30% w/w, from about 15% to about 25% w/w, or from about 4% to about 6% w/w.

### Starch

The viscoelastic mass may include a starch. As used herein, “starch” refers to any substance comprised of more than about 80%, 90%, 95%, or even 99% amylase and amylopectin by weight. Starches from various sources are known in the art. Suitable starches can be obtained from tuberous foodstuffs, such as potatoes, tapioca, and the like. Other suitable starches can be obtained upon grinding cereal grains such as corn, oats, wheat, milo, barley, rice, and others. The starch may be included in an amount of from about 0.1% to about 25% w/w of the viscoelastic mass, such as from about 1% to about 15% w/w or from about 5% to about 9% w/w.

### Antioxidants

The viscoelastic mass preferably includes an antioxidant. Examples of suitable antioxidants include alpha-tocopherol, alpha-tocopherol acetate, butylated hydroxytoluene (BHT), ascorbic acid, mixed tocopherols, propyl gallate, and mixtures thereof. The antioxidant may be included in an amount of from about 0.01% to about 0.3% w/w of the viscoelastic mass, such as from about 0.025% to about 0.2% w/w, or from about 0.05% to 0.15% w/w.

### Salts

The viscoelastic mass preferably includes one or more salts comprising mono- and/or divalent cations for proper gelation of the mass. Suitable sources of mono- and divalent cations include sodium, potassium or calcium salts such as sodium chloride, potassium chloride, calcium chloride, or potassium citrate, among others. Such a salt may be included in an amount of from about 0.01% to about 5% w/w of the viscoelastic mass, such as from about 0.1% to about 2.5% w/w or from about 0.4% to about 0.6% w/w.

### Water and Oil

The viscoelastic mass preferably includes water in an amount of from about 1% to about 50% w/w of the viscoelastic mass, such as from about 1% to about 30% w/w or about 5% to about 15% w/w. The viscoelastic mass preferably includes an oil in an amount of from about 1% to about 50% w/w of the viscoelastic mass, such as from about 1% to about 30% w/w or about 5% to about 15% w/w. Suitable oils include, for example, canola oil, corn oil, soybean oil, and vegetable oil, among others.

### Preservatives

The viscoelastic mass preferably includes a preservative to prevent or retard growth of microorganisms and fungi. Suitable preservatives include potassium sorbate, methylparaben, propylparaben, sodium benzoate, calcium propionate, or combinations thereof. A preferred preservative comprises a combination of potassium sorbate, methylparaben, and propylparaben. The preservatives may be included in an amount of from about 0.01% to about 2% w/w of the viscoelastic mass, such as from about 0.03% to about 1.5% w/w or from about 0.05% to about 1% w/w.

### Probiotics

The viscoelastic mass may include a probiotic microorganism. Probiotics are microorganisms which beneficially affect a host by improving its intestinal microbial balance. The beneficial effects of probiotic microorganisms include activation of the immune system, prevention of the bacterial overgrowth by pathogens, prevention of diarrhea, and/or restoration of intestinal flora. Research has shown (Trachoo, N. and Bourdeaux, C. “Therapeutic properties of probiotic bacteria.” Journal of Biological Sciences. 2006 6(1):202-208) that probiotics produce natural antibiotics, which can fight harmful bacteria, regulate and increase hormone levels, manufacture B group vitamins, particularly cyanocobalamin, biotin and folic acid, stimulate the immune system, reduce food intolerance, reduce inflammation, increase energy levels, and increase digestibility of food.

Newborn animals are born with a sterile gut and undeveloped immune system. It is believed that the beneficial intestinal commensal microflora is established at birth, and is something the mother passes on to her newborn with the mother's colostrum milk. Probiotics are of similar genera and species as these microflora except that they are provided to the animal in the form of a supplement. Animals that do not acquire beneficial microflora from the mother for various reasons, such as those that are orphaned or placed on milk replacers for other reasons are quite susceptible to picking up pathogenic organisms from their environment. These newborns are particularly susceptible to diseases at birth and for the next couple of months. The probiotics included in the viscoelastic mass of the present invention can help to establish the commensal microflora to treat or prevent such diseases in newborns and help to maintain good digestion, stimulate immunity, improve utilization of food, reduce intestinal upsets, improve health, and prevent the proliferation of some potential disease-causing pathogens.

A delicate balance of commensal microorganisms is typically already present in the gastrointestinal (GI) tract of healthy older animals. However, various stresses such as infections, diarrhea, surgery, the use of antibiotics, kenneling, or other events can cause an imbalance of the beneficial microorganisms. Bacterial, viral, or fungal infections, for example, can result in an imbalance of beneficial microflora and cause diarrhea when the pathogens outgrow the beneficial gut microflora. When antibiotics are used to fight these infections, not only are the pathogens killed, but the beneficial microflora can be killed as well. Changes in diet sometimes leads to diarrhea and can also change the balance of gut microflora as different nutrients in the food might be used by different microflora, thus leading to imbalances. Simpler stresses in pets from various sources such as traveling or kenneling can also result in imbalances of beneficial gut microflora. Probiotics can be administered in both the short term (i.e., several days) and the long term for older animals in these stressful situations to restore the balance of microflora. Probiotics can also be administered in both the short term and the long term for healthy older animals by providing a constant presence of the beneficial microorganisms in the GI tract, thereby helping to maintain health.

Suitable probiotics for including in the viscoelastic mass include but are not limited to microorganisms of the genera Aspergillus, Trichoderma, Bacillus, Bacteriodies, Bifidobacterium, Lactobacillus, Leuconostoc, Streptococcus, Pediococcus, Propionibacterium, Saccharomyces, Enterococcus, and Escherichia. Suitable species of these genera include but are not limited to Aspergillus oryzae, Aspergillus niger, Trichoderma longbranchiatum, Bacillus subtilus, Bacteriodies thetaiotaomicron, Bacteriodies longum, Bifidobacterium longus, Bifidobacterium infantis, Lactobacillus acidophilus, Lactobacillus acidophilus, Lactobacillus bulgaricus, Lactobacillus casei, Lactobacillus lactis, Lactobacillus plantarum, Lactobacillus rhamnosus, Lactobacillus sakei, Leuconostoc mesenteroides, Leuconostoc cremoris, Streptococcus diacetylactis, Streptococcus florentinus, Streptococcus thermophilus, Pediococcus acidilactici, Pediococcus pentosaceus, Propionibacterium freudenreichii, Saccharomyces boulardii, Enterococcus faecalis, Enterococcus faecium, Escherichia coli Nissle 1917. The probiotic may be included in a spray dried or freeze-dried form in an amount of from about 1 to about 1×10¹¹ CFU/g of the viscoelastic mass, such about 2.5×10⁸ CFU/g.

### Prebiotics

The viscoelastic mass may include a prebiotic. Prebiotics are indigestible carbohydrates, generally oligosaccharides, that resist hydrolysis by enzymes in the animal digestive tract and reach the colon undegraded to provide a carbohydrate source for commensal microorganisms. In this manner, prebiotics directly and selectively stimulate the growth and activity of certain commensal microflora that colonize the colon, such as Lactobacillus and Bifidobacterium. Prebiotics can either be delivered alone or combination with probiotics. The combination of prebiotics and probiotics are known as “synbiotics” (Sekhon, B. S. and Saloni, J. “Prebiotics, probiotics and synbiotics: an overview.” Journal of Pharmaceutical Education and Research. 2010 1:13-36). Synbiotics provide a synergistic effect to promote gastrointestinal health and well-being.

Suitable prebiotics for including in the viscoelastic mass include but are not limited to inulin, lactulose, lactitol, fructooligosaccharides, galactooligsaccharides, xylooligosaccharides, isomaltooligosaccharides, mannaoligosaccharides, lactosucrose, cereal fibers, soy oligosaccharides, raffinose, beet pulp, psyllium, cellulose, and gum arabic. The prebiotic may included in an amount of from about 0.1% to about 10% w/w of the viscoelastic mass, such as from about 0.5% to about 6% w/w or from about 1% to about 3% w/w.

### Enzymes

The viscoelastic mass may include one or more enzymes, such as enzymes that aid in the digestion of food. Animal feed and pet foods are typically composed of plant materials, cereals, and vegetable proteins which cannot be fully digested and utilized by the animals. The animals either do not have the required enzymes to fully digest the food or do not have enough of the required enzymes. In addition, many animal foods are heat processed, which can significantly reduce the enzyme concentrations in the food itself. As a result, many nutrients pass through animals undigested and unabsorbed. Many cereals, for example, have a proportion of their energy in the form of non-starch polysaccharides (NSPs), more commonly known as fiber. Adding certain enzymes to dog food increases digestion of the NSPs (Twomey L N, J R Pluske, J B Rowe, M Choct, W Brown, M F McConnell, D W Pethick. “The effects of increasing levels of soluble non-starch polysaccharides and inclusion of feed enzymes in dog diets on faecal quality and digestibility.” Animal Feed Science and Technology. 2003 108(1-4):71-82).

Administering digestive enzymes to animals by including them in the viscoelastic mass of the present invention enhances digestion of food and absorption of nutrients and thereby provides several benefits. These benefits include enhanced overall animal health, increased animal weight gain over a shorter period of time, and increased feed efficiencies.

Various enzymes that may be included in the viscoelastic mass of the present invention include carbohydrases, lipases, and proteases. Carbohydrases are enzymes capable of catabolizing carbohydrates. Examples of suitable carbohydrases include xylanases, glucanases such as beta-glucanase, glucosidases such as alpha-glucosidase (sucrase), galactosidases such as beta-galactosidases (lactase) and alpha-galactosidases, glycanases, amylases such as alpha-amylase, and cellulases. Lipases are enzymes capable of catabolizing lipids. Examples of suitable lipases include any of the various esterases that cleave fatty acids from other lipid or non-lipid moieties, among others. Proteases are enzymes capable of catabolizing proteins. Examples of suitable proteases include exopeptidases, such as aminopeptidases and carboxypeptidase A, among others. Other suitable proteases include endopeptidases, such as trypsin, chymotrypsin, pepsin, papain, and elastase, among others. Organisms from the genera Aspergillus (e.g., Aspergillus oryzae, Aspergillus niger), Trichoderma, Humicola, Candida, Rhizomucor and Thermomyces produce enzymes suitable for inclusion in the viscoelastic mass of the present invention. The enzymes may be obtained by purifying them from the fermentation broth of these microorganisms or by concentrating the broth itself.

Each individual enzyme may be included in an amount of from about 0.01 units/lb to about 500,000 units/lb of the viscoelastic mass, such as from about 1 unit/lb to about 100,000 units/lb, from about 1 unit/lb to about 500 units/lb, or from about 150 units/lb to about 200 units/lb. Definitions for the unit activity for the enzymes described herein are well-known in the art.

### Vitamins

The viscoelastic mass may include one or more vitamins. Vitamins are necessary for literally tens of thousands of different chemical reactions in the body. They often work in conjunction with minerals and enzymes to assure normal digestion, reproduction, muscle and bone growth and function, healthy skin and hair, clotting of blood, and the use of fats, proteins, and carbohydrates by the body. For example, vitamin E isomers (mixed tocopherols) are antioxidants that help protect animals from free radical damage. Vitamin deficiencies can occur in an animal if poor quality food is provided to the animal. Vitamin deficiencies can also occur if an animal is under stress. Ill or recovering animals that may have a poor appetite typically need a vitamin supplement since they are not receiving their daily requirements through the food they eat. Animals in other situations such as stress from travel, showing, training, hunting, breeding, or lactation can also benefit from vitamin supplementation. Older animals can also benefit from vitamin supplementation. Older animals tend to absorb fewer vitamins, minerals, and electrolytes through the intestinal tract, and lose more of them through the kidneys and urinary tract. Also, some older animals eat less (due to conditions such as oral disease) and may not receive their daily needs of vitamins and minerals. These same old animals are often the ones that will also be given solid medications to treat other conditions. Another issue that may increase the need for vitamin supplementation in animals is that commercial feeds typically involve a heating process that can destroy vitamins present in the feed. The viscoelastic mass of the present invention does not involve heat for manufacture and is therefore able to provide vitamins that are not degraded.

Any vitamin known in the art may be included in the viscoelastic mass of the present invention. Particular vitamins may be provided according to the nutritional requirements of the target animal. Suitable vitamins include both water soluble and/or fat soluble vitamins. Exemplary water soluble vitamins include any or all of the B vitamins (Vitamin B₁, B₂, B₃, B₄, B₅, B₆, B₇, B₈, B₉, B₁₀, B₁₁, and B₁₂) and/or Vitamin C. Exemplary fat soluble vitamins include Vitamin A, Vitamin D, Vitamin E, and Vitamin K. The fat-soluble vitamins may be provided as an element of oils utilized in the present invention, such as, for example, canola oil, corn oil, soybean oil, and vegetable oil. The amount of the vitamins included in the viscoelastic mass can be adapted to the specific needs of the target animal. As an example, each vitamin may be included in an amount of from about 0.001% to about 10% w/w of the viscoelastic mass, such as from about 0.01% to about 5% w/w or from about 0.5% to about 1% w/w.

### Amounts of Components

The amounts of each of the components in the viscoelastic mass may be varied from the amounts described herein depending upon the nature of the delivery drug, the weight and condition of the animal to be treated, and the unit dosage desired. Those of ordinary skill in the art will be able to adjust dosage amounts as required.

### Preparation

The individual ingredients in the composition of the invention are mixed together in a standard mixing apparatus. The dry powders are mixed initially. This is followed by the addition of liquid materials to create a soft dough that is easily pliable by hand. The materials are mixed until the dough composition has reached a satisfactory pliability texture level and no dry materials are present. The dough is then transferred to an extruder device hopper. The extruder device hopper feeds the soft dough through an extrusion port, and a knife blade chops the extruded dough composition to a desired length and weight. The cut pieces of the invention are subsequently packaged.

The extruded dough can form any cross-sectional shape depending on the extrusion port design. Suitable shapes include rectangles, squares, circles, triangles, or other specific shapes such as animal or bone shapes. A preferred cross-sectional shape is a thin rectangle. The extruded dough can also have any length, which is determined by the distance between the knife cuts as the dough leaves the extrusion port. The size of the final product may be varied depending on the size of the target animal and the size of the solid medication to be wrapped. In a preferred version of the invention, the viscoelastic mass takes the form of a sheet, i.e., having a depth less than about half the magnitude of the length and width, such as a depth less than about a quarter the magnitude of the length and width.

### Use

The viscoelastic mass can be used on its own as a treat or, if including probiotics, prebiotics, enzymes, and/or vitamins, as a nutritional supplement. In addition or alternatively, the viscoelastic mass can be disposed about a medication to conceal the medication and/or mask the taste of the medication. The viscoelastic mass can be wrapped or folded around the medication. Alternatively, the medication can be pushed into the viscoelastic mass such that the viscoelastic mass envelops the medication. The medication take any of several solid or semi-solid (gel) forms including pills, tablets, capsules, or boluses such that the medication forms a phase distinct from the viscoelastic mass when included therewith.

The viscoelastic mass can be administered, with or without additional medication, to any animal, including mammals or humans, in need of nutritional supplementation and/or a particular medication. Non-limiting examples of suitable animals include humans, dogs, cats, horses, cows, pigs, goats, and sheep, among others.

In one exemplary use, the viscoelastic mass is administered without additional medication to an animal on a regular basis. The animal will gain the nutritional advantages of the viscoelastic mass and will additionally be accustomed to consuming the mass. When it is time for medication to be given to the animal, the animal will be accustomed to consuming the viscoelastic mass and will more readily accept any medication included with the viscoelastic mass.

### EXAMPLE 1

Ingredients as found in Table 1 were mixed together by first mixing the dry materials in a mixing device followed by mixing in the liquid ingredients to create a base composition. The mixing resulted in a soft dough that was extruded through an extrusion device. The dye shape on the extruder resulted in a rectangle shape of 1 inch by 2 inches by ¼ inch of the composition. The extruded shaped material was packaged. Individual pieces of the invention were easily wrapped around solid tablets by hand. This resulted in the tablet being completely covered. The covered tablet was presented to a dog and was immediately consumed with no rejection of the tablet material. This indicated that the formula matrix was palatable to the dog that had previously rejected the tablet without wrapping the tablet.

TABLE 1
Base composition

| Ingredient | % w/w |
|---|---|
| Soy Flour | 9.80% |
| Soy Lecithin | 8.00% |
| Pregelatinized Starch | 7.00% |
| Dry Molasses | 14.00% |
| Soy Oil | 7.90% |
| Glycerine | 11.00% |
| Poultry Liver Powder | 12.00% |
| Potassium Sorbate | 0.50% |
| Methylparaben | 0.10% |
| Propylparaben | 0.10% |
| Sodium Chloride USP | 0.50% |
| Powder Sugar | 5.00% |
| Mixed Tocopherols | 0.10% |
| Whole Wheat Fine Flour | 15.50% |
| Water | 8.50% |

### EXAMPLE 2

Ingredients as found in Table 2 were mixed together by first mixing the dry materials in a mixing device followed by mixing in the liquid ingredients to create the base formulation with probiotics. The mixing resulted in a soft dough that was extruded through an extrusion device. The dye shape on the extruder resulted in a rectangle shape of 1 inch by 2 inches by ¼ inch of the composition. The extruded shaped material was packaged. Individual pieces of the invention were wrapped around solid tablets by hand. This resulted in the tablet being completely covered. The covered tablet was presented to a dog and was immediately consumed with no rejection of the tablet material. This indicated that the formula matrix containing probiotics in the form of Bacillus subtilis was palatable to the dog that had previously rejected the tablet without wrapping the tablet. The fecal content of the dog was analyzed for the presence of Bacillus subtilis before and after receiving the invention containing the probiotic Bacillus subtilis. No Bacillus subtilis was present prior to receiving the invention. After receiving the invention Bacillus subtilis was present in the fecal matter. These results indicate that Bacillus subtilis in the invention was able to survive passage through the GI tract of the dog and is consistent with the ability of the invention to provide health benefits to the animal that probiotics can provide.

TABLE 2
Base formulation with probiotic

| | % w/w |
|---|---|
| Ingredient | (except for B. subtilis) |
| Soy Flour | 9.80% |
| Soy Lecithin | 8.00% |
| Pregelatinized Starch | 7.00% |
| Dry Molasses | 14.00% |
| Soy Oil | 7.90% |
| Glycerin | 11.00% |
| Poultry Liver Powder | 12.00% |
| Potassium Sorbate | 0.50% |
| Methylparaben | 0.10% |
| Propylparaben | 0.10% |
| Sodium Chloride USP | 0.50% |
| Powder Sugar | 5.00% |
| Mixed Tocopherols | 0.10% |
| Whole Wheat Fine Flour | 15.50% |
| Water | 8.50% |
| Bacillus subtilis | 2.5 × 10⁸ CFU/g |

EXAMPLE 3

Ingredients as found in Table 3 were mixed together by first mixing the dry materials in a mixing device followed by mixing in the liquid ingredients to create the base formulation with probiotics and prebiotics. The mixing resulted in a soft dough that was extruded through an extrusion device. The dye shape on the extruder resulted in a rectangle shape of 1 inch by 2 inches by ¼ inch of the composition. The extruded shaped material was packaged. Individual pieces of the invention were wrapped around solid tablets by hand. This resulted in the tablet being completely covered. The covered tablet was presented to a dog and was immediately consumed with no rejection of the tablet material. This indicated that the formula matrix containing probiotics and prebiotics in the form of probiotic Bacillus subtilis and the prebiotic inulin was palatable to the dog that had previously rejected the tablet without wrapping the tablet.

TABLE 3
Base composition with probiotic and prebiotic

| | % w/w |
|---|---|
| Ingredient | (except for B. subtilis) |
| Soy Flour | 7.80% |
| Soy Lecithin | 8.00% |
| Pregelatinized Starch | 7.00% |
| Dry Molasses | 14.00% |
| Soy Oil | 7.90% |
| Glycerine | 11.00% |
| Poultry Liver Powder | 12.00% |
| Potassium Sorbate | 0.50% |
| Methylparaben | 0.10% |
| Propylparaben | 0.10% |
| Sodium Chloride USP | 0.50% |
| Powder Sugar | 5.00% |
| Mixed Tocopherols | 0.10% |
| Whole Wheat Fine Flour | 15.50% |
| Water | 8.50% |
| Inulin | 2.00% |
| Bacillus subtilis | CFU/g 2.5 × 10⁸ |

There are several variations which can be practiced in the scope of this invention. The invention may be provided in a variety of shapes and sizes as long as the invention is chewable and the sufficiently soft to trap medications when folded around solid medications.

There are many advantages to the composition for administering medication of this invention. Chiefly, by administering medication in a food treat, the pet owner does not need to force the medication down the pet's throat. Not only is this an easier way to administer medicine, but it safer and more pleasant for the owner and the pet. Another advantage is to provide health benefits from probiotics, prebiotics enzymes and vitamins on routine basis. The manufacturing process also allows for the probiotics, enzymes and vitamins to maintain viability and stability as no heating processes are present that normally degrade probiotics, prebiotics, enzymes and vitamins.

Any version of any component or method step of the invention may be used with any other component or method step of the invention. The elements described herein can be used in any combination whether explicitly described or not.

All combinations of method steps as used herein can be performed in any order, unless otherwise specified or clearly implied to the contrary by the context in which the referenced combination is made. As used herein, the singular forms “a,” “an,” and “the” include plural referents unless the content clearly dictates otherwise.

Unless explicitly stated otherwise, amounts of the individual components disclosed herein are to be understood as weight of the individual component per the total weight of the viscoelastic mass (i.e., “w/w” or “w/w of the viscoelastic mass”), such as percent weight of the individual component per the total weight of the viscoelastic mass (“% w/w of the viscoelastic mass”).

Unless explicitly stated otherwise, the amounts of the components disclosed herein refer to the total amounts of the type of component, even if the type of component comprises several different species of the type. For example, a preservative in an amount of from about 0.7% may comprise 0.7% potassium sorbate; 0.7% methylparaben; 0.7% propylparaben; or 0.1% methylparaben, 0.1% propylparaben, and 0.5% potassium sorbate; among other possibilities.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference in their entirety to the same extent as if each individual reference were specifically and individually indicated as being incorporated by reference. In case of conflict between the present disclosure and the incorporated references, the present disclosure controls.

While this invention may be embodied in many forms, what is described in detail herein is a specific preferred embodiment of the invention. The present disclosure is an exemplification of the principles of the invention is not intended to limit the invention to the particular embodiments illustrated. It is to be understood that this invention is not limited to the particular examples, process steps, and materials disclosed herein as such process steps and materials may vary somewhat. It is also understood that the terminology used herein is used for the purpose of describing particular embodiments only and is not intended to be limiting since the scope of the present invention will be limited to only the appended claims and equivalents thereof.

We claim:

1. A consumable composition for providing health benefits to an animal comprising:
a viscoelastic mass comprising:
a carrier base comprising components selected from the group consisting of a powder, an emulsifier, a starch, an oil, a softening agent, and water in a combination and in amounts effective to confer viscoelasticity to the viscoelastic mass; and
an effector component selected from the group consisting of a probiotic, a prebiotic, and an enzyme selected from the group consisting of a carbohydrase, a lipase, and a protease.

2. The composition of claim 1 comprising the probiotic, wherein the probiotic is a microorganism in a genus selected from the group consisting of Aspergillus, Trichoderma, Bacillus, Bacteriodies, Bifidobacterium, Lactobacillus, Leuconostoc, Streptococcus, Pediococcus, Propionibacterium, Saccharomyces, Enterococcus, Escherichia, and combinations thereof.

3. The composition of claim 1 comprising the probiotic in an amount of from about 0.01 to about 1×10¹¹ CFU/g of the viscoelastic mass.

4. The composition of claim 1 comprising the prebiotic, wherein the prebiotic is selected from the group consisting of inulin, lactulose, lactitol, a fructooligosaccharide, a galactooligosaccharide, a xylooligosaccharide, an isomaltooligosaccharide, a mannaoligosaccharide, a lactosucrose, a cereal fiber, a soy oligosaccharide, raffinose, beet pulp, psyllium, cellulose, gum arabic, and combinations thereof.

5. The composition of claim 1 comprising the prebiotic in an amount of from about 0.5% to about 6% w/w of the viscoelastic mass.

6. The composition of claim 1 comprising the carbohydrase.

7. The composition of claim 1 comprising the carbohydrase, wherein the carbohydrase is selected from the group consisting of a xylanase, a glucanase, and an amylase.

8. The composition of claim 1 comprising the enzyme, wherein the enzyme is from fermentation broth of a microorganism in a genus selected from the group consisting of Aspergillus, Trichoderma, Humicola, Candida, Rhizomucor, and Thermomyces.

9. The composition of claim 1 comprising the enzyme in an amount of from about 1 unit/lb to about 500 units/lb of the viscoelastic mass.

10. The composition of claim 1 wherein the viscoelastic mass further comprises a vitamin selected from the group consisting of a B vitamin, vitamin C, vitamin A, vitamin E, vitamin D, and vitamin K.

11. The composition of claim 1 wherein the viscoelastic mass further comprises mixed tocopherols.

12. The composition of claim 1 wherein the viscoelastic mass further comprises a vitamin in an amount of from about 0.01% to about 5% w/w of the viscoelastic mass.

13. The composition of claim 1 comprising the powder, wherein the powder comprises a plant powder and an animal powder.

14. The composition of claim 1 comprising the emulsifier, wherein the emulsifier comprises lecithin in an amount of from about 4% to about 16% w/w of the viscoelastic mass.

15. The composition of claim 1 comprising the softening agent, wherein the softening agent comprises glycerol in an amount of from about 9% to about 14% w/w of the viscoelastic mass.

16. The composition of claim 1 wherein the viscoelastic mass further comprises a preservative in an amount of from about 0.01% to about 3% w/w of the composition, wherein the preservative is selected from the group consisting of potassium sorbate, methylparaben, and propylparaben.

17. The composition of claim 1 wherein the viscoelastic mass further comprises a flavoring.

18. The composition of claim 1 wherein the viscoelastic mass is in the form of a pliable sheet.

19. The composition of claim 1 further comprising a solid or gel disposed within the viscoelastic mass and forming a distinct phase within the viscoelastic mass.

20. The composition of claim 1 further comprising a solid or gel, wherein the viscoelastic mass is in the form of a pliable sheet, and the viscoelastic mass is rolled or folded about the solid or gel.
